(19) United States
(12) Patent Application Publication
(10) Pub. No.: US 2008/0051723 A1
(43) Pub. Date: Feb. 28, 2008

(54) Device for storing and transporting microneedles and a method for delivering active substances with the aid of such a device

(76) Inventors: Franz Laermer, Weil Der Stadt (DE); Michael Stumber, Korntal-Muenchingen (DE); Dick Scholten, Stuttgart (DE); Christian Maeurer, Leonberg (DE); Julia Cassemeyer, Reutlingen (DE)

Correspondence Address: KENYON & KENYON LLP, ONE BROADWAY, NEW YORK, NY, 10004, US

(21) Appl. No.: 11/821,994

(22) Filed: Jun. 25, 2007

(30) Foreign Application Priority Data
Jun. 23, 2006 (DE) 102006028782.7

(51) Int. Cl. A61M 5/162

(52) U.S. Cl. 604/191; 604/507

(57) ABSTRACT

A device for storing and transporting microneedles and a method for delivering active substances with the aid of such a device. In the device for storing and transporting microneedles, a reservoir is provided which contains an active substance for delivery by the microneedles and the microneedles are situated within a package forming the reservoir.

### BACKGROUND INFORMATION

The concept of delivering medication, i.e., medical or biochemical active substances, with the aid of microneedles has been pursued in many variants. In principle, there are multiple important criteria to be observed here. One of the important criteria is, among others, the mechanical stability and biocompatibility of the microneedles, but also the possibility to store adequate amounts of active substances and deliver them via the microneedles.

If only very small amounts of active substances are to be delivered, an attempt may be made to coat the microneedles only on the outside. Typically relatively small amounts of active substances are sufficient, for example, in the case of vaccines. Alternatively, structures such as cavities or depressions may be situated within the microneedles for storing active substances.

U.S. Pat. No. 6,334,856, for example, describes microneedles having reservoirs for storing active substances. The reservoir is preferably situated underneath the needles on the substrate side and connected to the interior of the microneedles via internal ducts such as holes, whereby active substances are removed from the reservoir and delivered via microneedles to a body to be treated.

Often, however, the capacity either in the interior of the microneedles or in the reservoirs in the substrate is insufficient for storing adequate amounts of active substances for delivery. In addition, in the case of such storage approaches, sealing problems often occur. Consequently, a slow but not negligible outflow or evaporation of the active substances (leakage problem) is possible during storage.

An object of the present invention is to provide a device and a method which would eliminate or at least mitigate the above-mentioned disadvantages.

### SUMMARY OF THE INVENTION

The present invention has the advantage over the related art that it allows a relatively large amount of active substance to be stored. This allows rapid and convenient delivery even of large amounts of active substance.

At the same time, sterile storage of the microneedles is ensured. Furthermore, the above-described leakage problem is avoided; therefore, the microneedles may also be safely transported.

### BRIEF DESCRIPTION OF THE DRAWINGS

FIG. 1 shows an exemplary embodiment of a device for storing and transporting microneedles in a schematic sectional view.

FIG. 2 shows an exemplary embodiment of a method for delivering active substances with the aid of a device according to FIG. 1.

### DETAILED DESCRIPTION

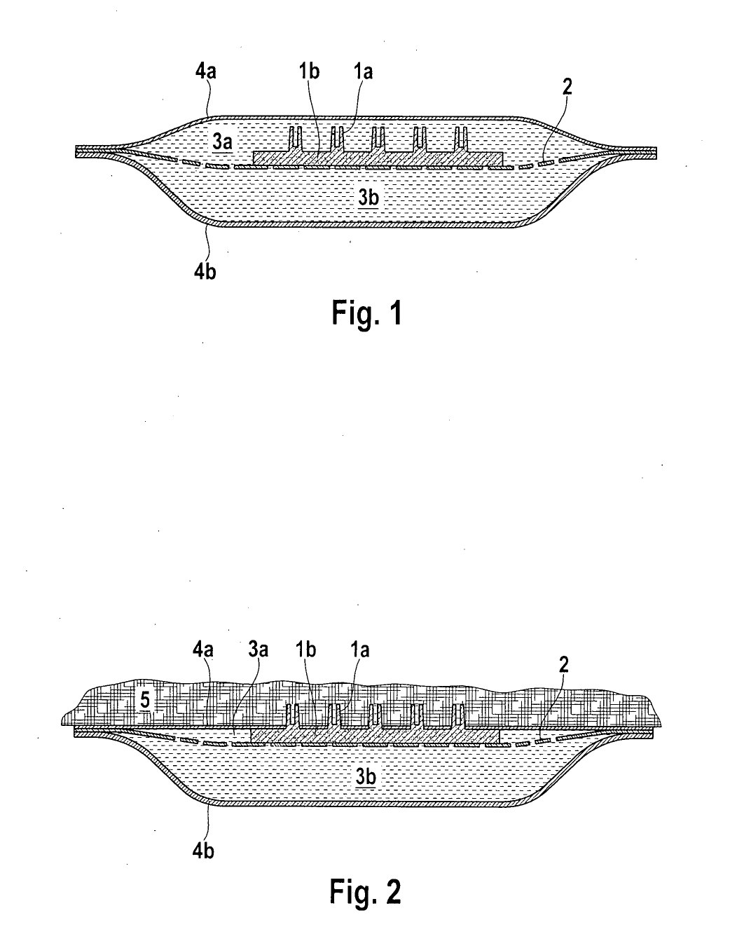

A device is provided for storing and transporting microneedles 1a,1b, a reservoir 3a,3b being provided which contains an active substance for delivery by microneedles 1a,1b and microneedles 1a,1b being situated within a package 4a,4b forming reservoir 3a,3b (FIG. 1). Due to the fact that reservoir 3a,3b is not provided as a subregion of microneedles 1a,1b, but is formed externally by a package 4a,4b, large amounts of active substances may actually be stored. At the same time, microneedles 1a,1b are situated within package 4a,4b, i.e., microneedles 1a,1b are provided in the middle of reservoir 3a,3b.

The most diverse specific embodiments of microneedles 1a,1b itself are known from the related art. They are advantageously arranged in an array and have needle tips 1a on a substrate 1b. Microneedles 1a,1b are made, as needed, of a semiconductor material such as silicon, but also of plastic or metal.

In this exemplary embodiment, microneedles 1a,1b are applied to a substrate 2 that is permeable to the active substance. Substrate 2 may be implemented as a thin film having small openings. Substrate 2, which is permeable to the active substance, extends through reservoir 3a,3b, preferably through the entire reservoir 3a,3b. Reservoir 3a,3b is subdivided by substrate 2 into a first reservoir region 3a having microneedles 1a,1b, and a second reservoir region 3b without microneedles 1a,1b.

Package 4a,4b itself is formed by an elastic film 4a, perforatable by microneedles 1a,1b, and an outer layer 4b, which are connected at their edge areas. Therefore, in this case, the area between film 4a and outer layer 4b within the connected edge areas define the reservoir. In turn, microneedles 1a,1b are supported by substrate 2 and are covered by elastic film 4a, which protects microneedles 1a,1b during transport and storage and, in addition, keeps microneedles 1a,1b and the active substance sterile. Furthermore, package 4a,4b, in particular film 4a, protects against leakage or evaporation of the active substance.

Elastic film 4a and/or substrate 2 preferably have adhesive properties, at least in their edge areas; this makes it possible to glue film 4a and substrate 2 together by exerting a pressure.

A method for delivering active substances with the aid of a device having microneedles 1a,1b, which are situated within a package 4a,4b forming reservoir 3a,3b for active substances is now elucidated with reference to FIG. 2. The method includes the following steps:
applying the device to a skin 5 to be treated,
exerting a pressure on the device, whereby microneedles 1a,1b perforate package 4a,4b and penetrate skin 5,
transporting the active substance from reservoir 3a,3b into skin 5 via microneedles 1a,1b.

As pressure is exerted on the device, package 4a,4b functions as a cover of the remaining structure and laterally encloses microneedles 1a,1b in such a way that leakage is further minimized and the active substance exits only through microneedles 1a,1b and directly under the outermost skin layers. The active substance transport is ensured by a diffusion process from the reservoir into the skin, i.e., into the body.

When a package 4a,4b having an elastic film 4a and an outer layer 4b, which are connected at their edge areas, and microneedles 1a,1b are used, which are mounted on a substrate 2 permeable to the active substance, extending through reservoir 3a,3b, when pressure is exerted on the device, substrate 2 is pressed against elastic film 4a and microneedles 1a,1b perforate, i.e., puncture, film 4a. The active substance may now diffuse into skin 5 through microneedles 1a,1b.

In another specific embodiment, film 4a and/or substrate 2 having adhesive properties at least in their edge areas are used, whereby, when pressure is exerted on the device, film 4a and substrate 2 are glued together, securing microneedles 1a,1b and holding them in place. Microneedles 1a,1b thus become stably and unmovably clamped in between film 4a and substrate 2 and thus ensure controlled delivery of the active substance.

In summary, it may be stated that the device according to the above description may be used as a drug delivery patch. In general, the device according to the present invention is suitably used in (bio)chemical, medical, or clinical areas. A simple and, at the same time, reliable application of microneedles 1a,1b for delivering even large amounts of active substances is made possible.

What is claimed is:

1. A device for storing and transporting microneedles comprising:
a package for containing the microneedles, the package forming a reservoir, the reservoir containing an active substance for delivery by the microneedles.

2. The device according to claim 1, wherein the microneedles are mounted on a substrate permeable to the active substance.

3. The device according to claim 2, wherein the substrate permeable to the active substance extends through the reservoir, subdividing the reservoir into a first reservoir region having microneedles, and a second reservoir region without microneedles.

4. The device according to claim 2, wherein the package is formed by an elastic film, perforatable by the microneedles, and an outer layer, which are connected to each other at their edge areas.

5. The device according to claim 4, wherein at least one of the elastic film and the substrate has adhesive properties, at least in their edge areas, which makes it possible to glue the elastic film and the substrate together by exerting a pressure.

6. The device according to claim 1, wherein the microneedles are situated in an array and have needle tips on a substrate.

7. The device according to claim 1, wherein the microneedles are made of at least one of silicon, plastic, and metal.

8. A method for delivering an active substance with the aid of a device having microneedles, which are situated within a package forming a reservoir for the active substance, the method comprising:
applying the device to a skin to be treated;
exerting a pressure on the device, so that the microneedles perforate the package and penetrate the skin; and
transporting the active substance from the reservoir into the skin via the microneedles.

9. The method according to claim 8, wherein the package having an elastic film and an outer layer, which are connected at their edge areas, and the microneedles are used, which are mounted on a substrate permeable to the active substance and extending through the reservoir, wherein, when pressure is exerted on the device, the substrate is pressed against the elastic film and the microneedles perforate the elastic film.

10. The method according to claim 9, wherein at least one of the elastic film and the substrate having adhesive properties at least in their edge areas is used, wherein, when pressure is exerted on the device, the elastic film and the substrate are glued together, securing the microneedles and holding the microneedles in place.
